(19) United States
(12) Patent Application Publication
(10) Pub. No.: US 2005/0233005 A1
(43) Pub. Date: Oct. 20, 2005

(54) Use of glycosaminoglycans and zinc derivatives for insect bites, stings and the like

(76) Inventor: Mitchell Kyle, Montreal (CA)

Correspondence Address: Intellectual Property Department;DEWITT ROSS & STEVENS, S.C., Firstar Financial Centre, 8000 Excelsior Drive Suite 401, Madison, WI, 53717-1914, US

(21) Appl. No.: 10/827,127

(22) Filed: Apr. 19, 2004

(51) Int. Cl. A61K 31/727; A61K 33/32

(52) U.S. Cl. 424/641; 514/56

(57) ABSTRACT

A composition of glycosaminoglycans and zinc derivatives and its application to accelerate wound healing and to reduce pruritus resulted from insect bites, stings and the like are disclosed. Effective amount of zinc derivatives in the range of 1-20 mg/g is admixed with effective amount of glycosaminoglycan, such as heparin derivative, in the range of 100-300 USP units/g in a gel for topical application on the affected area. Suitable non-medicinal carriers selected from the group consisting of carboxymethylcellulose, glycerin, methylparaban, polysorbate, propylparaben and water may be added to the composition gel.

### FIELD OF THE INVENTION

The invention relates to a composition of glycosaminoglycans and zinc derivatives and its use to effectively accelerate wound healing and reduce pruritus resulted from insect bites, stings and the like.

### BACKGROUND OF THE INVENTION

Insect bites, such as caused by mosquitoes, can lead to an allergic reaction and pruritus (itching), which is mediated in part by the production of antisaliva IgE antibodies and histamine (Karppinen et al., 2002). Antihistamines such as cetirizine and ebastine administered propylatically have been demonstrated to significantly decrease pruritus caused by mosquito bites (Karppinen et al., 2002). Topical application of calamine lotion (which usually contains zinc oxide and calamine) at the site of the insect bite, sting or the like is also indicated for symptomatic relief of mild pruritus (World Health Organization, 1997). Topical application of glucocorticosteroids such as hydrocortisone are used to relieve pruritus and in the treatment of inflammatory skin diseases (Wyatt et al., 2001; World Health Organization, 1997), but are not commonly used for the treatment of insect bites, stings and the like. In short, current methods for treatment of insect bites, stings and the like are directed towards minimizing/controlling the allergic/inflammatory reaction and symptomatic relief of pruritus.

Use of glycosaminoglycans, such as hyaluronic acid, as a single active ingredient to treat ailments, including insect bites is known (see U.S. Pat. Nos. 5,650,157, 6,103,704, 6,576,650 and U.S. patent application Publication No. 2003/0027833). Likewise, U.S. Pat. Nos. 4,879,282 and 5,037,810 teach the use of heparin for relieving pruritus. These prior art tend to address the most conspicuous aspect of insect bites, namely itching. They do not seem to deal with the wound healing aspect of the ailment. Thus, it is beneficial to have a composition which can be used to promote insect bite wound healing as well as to reduce pruritus.

This invention teaches wound healing of insect bites, stings and the like by topically applying effective amounts of glycosaminoglycans and zinc derivatives mixtures to the injured site for accelerated healing.

### SUMMARY OF THE INVENTION

It is an object of the invention to provide for an effective compound for promoting effective healing of insect bites, stings and the like on humans. According to one aspect of the invention, it provides a composition to promote wound healing and to reduce pruritus resulted from insect bites, comprising a therapeutically effective amount of zinc derivative and glycosaminoglycan admixed with non-medicinal carriers.

It is another object of the invention to provide for a method to promote wound healing and reduce pruritus resulting from insect bites. According to another aspect of the invention, it provides a method for promoting wound healing and reducing pruritus resulted from insect bites, comprising applying topically to the affected area an effective amount of zinc derivative and glycosaminoglycan composition admixed with non-medicinal carriers.

### DETAILED DESCRIPTION OF THE PREFERRED EMBODIMENT

In order to understand the mechanism of action of the present invention which uses glycosaminoglycans, such as heparin, and zinc derivatives for promoting wound healing and reducing pruritus of insect bites, stings and the like, the pharmacological characteristics of each of these compounds must first be understood.

Cationic Zinc

Zinc (Zn) is a naturally occurring trace element generally considered to be a non-toxic metal (Walsh et al., 1994). In its divalent cationic form, Zn²⁺ can be found in virtually every mammalian cell. Zn²⁺ is critical for the activity of a number of enzymes and proteins and is necessary for DNA synthesis, cell division and gene expression (Agren et al., 2001; Prasad et al., 1991). To maintain human health, Zn²⁺ must be consumed through dietary intake; indeed, Zn²⁺ deficiency resulting from either insufficient intake or diseased conditions (e.g. chronic renal failure) can lead to clinical symptoms (Prasad et al., 1991).

Zinc derivatives, primarily zinc oxide, are currently used in skin ointments, baby and skin creams, toothpaste, deodorants and sunscreens (SCCNFP report on Zinc Oxide, 2003). Toxicological studies using topical applications and oral administration of zinc derivatives (zinc oxide or zinc sulphate) in both animal models and humans indicate little or no toxicity (SCCNFP report on Zinc Oxide, 2003). Furthermore, application of a zinc oxide-containing ointment (2.68 g daily dose) to the skin of humans suffering from psoriasis did not significantly elevate plasma levels of Zn²⁺ (Derry et al., 1983). Based on currently available toxicological and pharmacological studies and extensive topical use of cationic zinc, topical application of zinc derivatives for the treatment of insect bites, stings and the like is expected to be safe and well-tolerated.

Heparin

A glycosaminoglycan, heparin is a polymer composed of alternating N-acetyl-glucosamine residues and D-glucoronic acid (reviewed in Bourin and Lindahl, 1993). The glucosamine residues can be deacetylated and sulfated on the amine residue on C2 or can be O-sulfated at the C3 and C6 position. The D-glucoronic acid residue can epimerize to L-iduronic acid and both glucoronic and iduronic acid residues can be O-sulfated at the C-2 position. Heparin derives its name from its high abundance in liver and can be found in tissues that contain mast cells (reviewed in Majerus and Tollefsen, 2001). Heparin is a potent anticoagulant with a rapid onset of action and is widely used clinically to treat venous thrombosis, pulmonary embolism, unstable angina, myocardial infarction and other vascular conditions requiring anticoagulant activity (reviewed in Majerus and Tollefsen, 2001). It is administered parenterally or by subcutaneous injection, with daily doses ranging from 10,000 to 40,000 USP units for the majority of clinical applications related to anticoagulant activity (Majerus and Tollefsen, 2001).

Other Glycosaminoglycans

Hyaluronic acid (HA), a polymer composed of the repeating disaccharide motif β-1,4-glucuronic acid-β1,3-N-acetyl glucosamine, is found in most vertebrate tissues (reviewed in Noble, 2002). This hygroscopic polymer in its high molecular weight form is a component in the extracellular matrix surrounding cells and is important for tissue structural integrity (Noble, 2002). The intra-articular administration of HA is currently employed for the treatment of osteoarthritis (reviewed in Moreland, 2003). Due to its viscoelasticity, HA is used during ophthalmic surgery (reviewed in Chen and Abatangelo, 1999).

Wound Healing

Wound healing following tissue injury can be divided into 4 sequential and overlapping processes: hemostasis, inflammation, proliferation and tissue remodelling (reviewed in Brissett and Hom, 2003). Blood clotting during hemostatis leads to the release of growth factors from platelets, hemostatic factors and inflammatory mediators such as cytokines (reviewed in Werner and Grose, 2002; Brissett and Hom, 2003). During the inflammatory phase, neutrophils phagocytose bacteria and cell debris (reviewed in Harding et al., 2002; Werner and Grose, 2002; Brissett and Hom, 2003). Neutrophils are subsequently replaced by macrophages. In the proliferative phase of wound healing, growth factors involved in angiogenesis and epithelialization such as fibroblast growth factor (FGF), vascular endothelial growth factor, transforming growth factor β and α, platelet-derived growth factor, epidermal growth factor, tumor necrosis factor and interferon, are released from macrophages and platelets (reviewed in Harding et al., 2002; Werner and Grose, 2002; Brissett and Hom, 2003). Fibroblasts migrate from the surrounding tissue, proliferate, deposit extracellular matrix proteins (such as collagen, elastin and integrins) and eventually transform into myofibroblasts (reviewed in Harding et al., 2002; Werner and Grose, 2002; Brissett and Hom, 2003). Keratinocytes proliferate at the edge of the injury and migrate across the wound bed. Wound remodelling involves increased collagen cross-linking and the destruction of excess collagen by collagenase released from myofibroblasts and macrophages (reviewed in Harding et al., 2002; Werner and Grose, 2002; Brissett and Hom, 2003).

Modulation of Wound Healing by Zinc Derivatives

Zinc oxide creams are commonly used for the treatment of diaper rash. Treatment of oral herpes with a zinc oxide/glycine preparation resulted in a shorter duration of cold sores and reduced blistering, soreness, itching, and tingling (Godfrey et al., 2001).

Zinc oxide is a common component of bandages applied to wounds (Agren et al., 2001). Studies have demonstrated that zinc oxide promotes epithelialization during wound repair although the mechanism for this process has yet to be elucidated (Agren, 1990; Rittenhouse, 1996; Agren et al., 2001). Topical application of zinc oxide to porcine wounds leads to enhanced release of growth factors (Tarnow et al., 1994; Agren et al., 2001). In addition, zinc oxide modulates the activity of zinc-dependent matrix metalloproteinases (Agren, 1999; Agren et al., 2001; Santos et al., 2001) which could facilitate keratinocyte migration during wound repair. This modulation by zinc oxide of growth factor release and matrix metalloproteinase activity is postulated to be involved in the epithelialization that occurs during wound repair. The effects of zinc oxide on wound healing are presumed to be the result of increased concentrations of zinc at the site of the injury (Agren et al., 1991). Hence, any zinc derivative that enhances the concentration of zinc at the site of an injury, such as an insect bites, stings and the like, will be beneficial to wound healing.

Modulation of Wound Healing by Heparin

The ability of heparin to assist in the healing of wounds, especially burn wounds (Saliba et al., 1973; reviewed in Saliba 1997, 2001) is documented, although the mechanism is unclear. Many growth factors, such as acidic and basic FGF, implicated in wound healing bind heparin with high affinity (Majerus and Tollefson, 2001). Binding to heparin results in stabilization and activation of these growth factors (Kratz et al., 1997) which are implicated in epithelialization during wound healing. The binding of heparin to growth factors may be responsible for enhanced wound healing. Heparin derivatives are thus expected to be effective for the treatment of insect bites, stings and the like.

Modulation of Wound Healing by Other Glycosamynoglycans

Topical application of HA to animals has been shown to accelerate skin wound healing (reviewed in Chen and Abatengelo, 1999). HA in wound dressings has been shown to be beneficial for the healing of diabetic foot wounds (Vazquez et al., 2003). The high quantity of HA in fetal when compared to adult tissues is believed to be linked to scarless wound healing observed in fetal tissues (reviewed in Chen and Abatengelo, 1999). The precise mechanism whereby HA assists in wound healing remains to be determined.

While there exist prior art which teaches treatment of insect bites, stings and the like by applying either zinc and/or zinc derivatives or glycosaminoglycans such as heparin alone, there has no reported discovery of combining the medicinal synergies of these two compounds to treat insect bites.

Combination of Zinc Derivatives and Glycosaminoglycans

Based on the above understanding, the combination of heparin (or other equivalent glycosaminoglycans) and zinc derivatives in a topical application should, in theory, enhance wound repair. The inventor of the present invention discovered and proved that this is, in fact, the case. Both heparin and zinc derivatives enhance epithilialization by different proposed mechanisms and, when mixed, interact in an additive or synergistic fashion to enhance wound repair. Therefore, combined use of a glycosaminoglycan, such as heparin, with a zinc derivative has significant advantages with respect to wound healing after an injury such as insect bites, stings and the like, when compared to the use of either component alone. In addition, zinc derivatives, such as zinc oxide, reduce itching. Unlike other current treatments for treatment of insect bites, stings and the like which are primarily aimed towards minimizing/controlling the allergic/inflammatory reaction and symptomatic relief of pruritus, the present invention is directed towards accelerating wound healing along with a reduction of pruritus. Thus, the use of both a glycosaminoglycan such as heparin with a zinc derivative such as zinc oxide is essential in order to effectively treat insect bites, stings and the like.

Formulation of Heparin and Zinc derivatives

According to the present invention, it has been found that applying zinc derivatives topically in the range of 1-20 mg/g of the gel and heparin derivatives (such as sodium heparin) topically in the range of 100-300 USP units/g of the gel provides effective therapeutic amount for treating insect bites, stings and the like on humans. Preferably, the optimal dosage used in the treatment is 5 mg/g of zinc derivatives and 160 USP units/g of heparin of the gel.

Based on currently available toxicological data (SCCNFP report on Zinc Oxide, 2003), the stated dosage range for zinc derivatives is safe and well tolerated. Likewise, the identified dosage range of heparin will not result in significant elevations in plasma heparin levels, hence will not lead to increased bleeding times, and hence will be safe and well-tolerated by the users. These two active ingredients will be applied topically to the skin at the site of the insect bite, sting or the like, in a gel formulation. Suitably, pharmaceutically acceptable carriers may be added to the application gel. Examples of non-medicinal carriers can be selected from the group consisting of carboxymethylcellulose, glycerin, methylparaban, polysorbate, propylparaben and water.

The preferred embodiment of the present invention will be further described by the following examples.

### EXAMPLE 1

The female black fly bite creates a hole through which blood is then sucked up. This process may initially go unnoticed by the victim because of the injected chemicals which are reported to contain toxins, painkillers and anticoagulants. The localized area of the wound then swells and becomes painful and extremely itchy. This condition remains with the victim for several days if left untreated.

Topical application with heparin sodium and zinc derivative in the preferred dosage of 160 USP units/g and 5 mg/g was initiated to a number of victims suffering from wounds resulted from these bites and which were in different time durations ranging from being bitten immediate to 12 hours or longer. In all cases the pain and itching relief was prompt, swelling in the wound area remarkably disappeared after one application.

### EXAMPLE 2

A four-year-old girl was playing in the sand on a lakefront beach and was stung by a large wasp in the right shoulder area. The swelling of the sting area was immediate and approximated the size of a dime. One topical application with heparin and zinc derivative in the preferred dosage of 160 USP units/g and 5 mg/g was administered within minutes of the sting. Further swelling, redness, aching or pain was not evident after a single application.

### EXAMPLE 3

A 67-year-old man was on a golf outing and accidentally closed his left hand over a small member of the wasp family. He was stung in the upper knuckle area of the third finger resulting in swelling, redness and itching. One topical application using the preferred dosage of 160 USP units/g and 5 mg/g of heparin sodium and zinc derivative promptly relieved the itching, pain and swelling with no further discomfort.

### EXAMPLE 4

A 43-year-old woman awoke with several large swollen red and itchy welts caused by an unknown bite. One topical application of heparin-zinc in the preferred dosage of 160 USP units/g and 5 mg/g quickly reduced the swelling with the redness and itching disappearing.

It is to be understood that the embodiments and variations shown and described herein are merely illustrative of the principles of this invention and that various modifications may be implemented by those skilled in the art without departing from the scope and spirit of the invention.

List of Prior Art Literatures

Agren M S (1990) Studies on zinc in wound healing. Acta Derm Venereol Suppl (Stockh) 154:1-36.
Agren M S (1999) Zinc in wound repair. Arch Dermatol 135:1273-1274.
Agren M S, Krusell M, Franzen L (1991) Release and absorption of zinc from zinc oxide and zinc sulfate in open wounds. Acta Derm Venereol 71:330-333.
Agren M S, Steenfos H H, Tarnow P, Jansson J O (2001) Zinc oxide augments engogenous expression of insulin-like growth factor-I (IGF-I) and activates matrix metalloproteinases (MWPs) in wounds. EWMA Journal 1:1-3.
Bourin M C, Lindahl U (1993) Glycosaminoglycans and the regulation of blood coagulation. Biochem J 289 (Pt 2):313-330.
Brissett A E, Hom D B (2003) The effects of tissue sealants, platelet gels, and growth factors on wound healing. Curr Opin Otolaryngol Head Neck Surg 11:245-250.
Chen W Y, Abatangelo G (1999) Functions of hyaluronan in wound repair. Wound Repair Regen 7:79-89.
Derry J E, McLean W M, Freeman J B (1983) A study of the percutaneous absorption from topically applied zinc oxide ointment. J Parenter Enteral Nutr 7:131-135.
Godfrey H R, Godfrey N J, Godfrey J C, Riley D (2001) A randomized clinical trial on the treatment of oral herpes with topical zinc oxide/glycine. Altern Ther Health Med 7:49-56.
Harding K G, Morris H L, Patel G K (2002) Science, medicine and the fiture: healing chronic wounds. Bmj 324:160-163.
Karppinen A, Kautiainen H, Petman L, Burri P, Reunala T (2002) Comparison of cetirizine, ebastine and loratadine in the treatment of immediate mosquito-bite allergy. Allergy 57:534-537.
Kratz G, Arnander C, Swedenborg J, Back M, Falk C, Gouda I, Larm O (1997) Heparin-chitosan complexes stimulate wound healing in human skin. Scand J Plast Reconstr Surg Hand Surg 31:119-123.
Majerus P W, Tollefsen D M (2001) Anticoagulant, thrombolytic, and anti-platelet drugs. In: Goodman & Gilman's The pharmacological basis of therapeutics, 10 Edition (Hardman J G, Limbird L E, Goodman Gilman A, eds), pp 1519-1538. New York: McGraw-Hill.
Moreland L W (2003) Intra-articular hyaluronan (hyaluronic acid) and hylans for the treatment of osteoarthritis: mechanisms of action. Arthritis Res Ther 5:54-67.
Noble P W (2002) Hyaluronan and its catabolic products in tissue injury and repair. Matrix Biol 21:25-29.
Prasad AS (1991) Discovery of human zinc deficiency and studies in an experimental human model. Am J Clin Nutr 53:403-412.
Rittenhouse T (1996) The management of lower-extremity ulcers with zinc-saline wet dressings versus normal saline wet dressings. Adv Ther 13:88-94.
SCCNFP: The Scientific Committee on Cosmetic Products and Non-Food Products intended for Consumers (2003) Evaluation and opinion on: Zinc oxide. Colipa n° S 76.
Saliba M J, Jr. (1997) The effects and uses of heparin in the care of burns that improves treatment and enhances the quality of life. Acta Chir Plast 39:13-16.
Saliba M J, Jr. (2001) Heparin in the treatment of burns: a review. Burns 27:349-358.
Saliba M J, Jr., Dempsey W C, Kruggel J L (1973) Large burns in humans. Treatment with heparin. Jama 225:261-269.
Santos M C, Souza A P, Gerlach R F, Tabchoury C M, Line S R (2001) Inhibition of human gelatinases (matrix metalloproteinase-2 and matrix metalloproteinase-9) activity by zinc oxide: a possible mechanism to enhance wound healing. Br J Dermatol 145:854-855.
Tarnow P, Agren M, Steenfos H, Jansson J O (1994) Topical zinc oxide treatment increases endogenous gene expression of insulin-like growth factor-1 in granulation tissue from porcine wounds. Scand J Plast Reconstr Surg Hand Surg 28:255-259.
Vazquez J R, Short B, Findlow A H, Nixon B P, Boulton A J, Armstrong D G (2003) Outcomes of hyaluronan therapy in diabetic foot wounds. Diabetes Res Clin Pract 59:123-127.
Walsh C T, Sandstead H H, Prasad A S, Newberne P M, Fraker P J (1994) Zinc: health effects and research priorities for the 1990s. Environ Health Perspect 102 Suppl 2:5-46.
Werner S, Grose R (2003) Regulation of wound healing by growth factors and cytokines. Physiol Rev 83:835-870.
World Health Organization. (1997) WHO model prescribing information: drugs used in skin diseases. Geneva: World Health Organilzation.
Wyatt E L, Sutter S H, Drake L A (2001) Dermatological pharmacology. In: Goodman & Gilman's The pharmacological basis of therapeutics, 10 Edition (Hardman J G, Limbird L E, Goodman Gilman A, eds), pp 1795-1819. New York: McGraw-Hill.

1-13. (canceled)

14. A composition for treating insect bites and stings, comprising a therapeutically effective amount of zinc oxide and sodium heparin admixed with pharmaceutically acceptable nonmedicinal carriers selected from the group consisting of carboxymethylcellulose, glycerin, polysorbate and water, wherein said effective amount for zinc oxide is in the range of 1-20 mg/g and for sodium heparin is in the range of 100-300 USP units/g of said composition.

15. The composition according to claim 14, wherein said effective amount for zinc oxide is 5 mg/g and for sodium heparin is 160 USP units/g of said composition.

16. A method for treating insect bites and stings, comprising applying topically to the affected area an effective amount of zinc oxide and sodium heparin admixed with pharmaceutically acceptable non-medicinal carriers selected from the group consisting of carboxymethylcellulose, glycerin, polysorbate and water, wherein said effective amount for zinc oxide is in the range of 1-20 mg/g and for sodium heparin is in the range of 100-300 USP units/g of the admixture.

17. The method according to claim 16, wherein said effective amount for zinc oxide is 5 mg/g and for sodium heparin is 160 USP units/g of said admixture.

18. Use of zinc oxide and sodium heparin admixed with pharmaceutically acceptable non-medicinal carriers selected from the group consisting of carboxymethylcellulose, glycerin, polysorbate and water, for treating insect bites and stings on human by applying topically to the affected area an effective amount of said zinc oxide and sodium heparin admixture, wherein said effective amount for zinc oxide is in the range of 1-20 mg/g and for sodium heparin is in the range of 100-300 USP units/g of said admixture.
